(19) United States
(12) Patent Application Publication
(10) Pub. No.: US 2017/0281835 A1
(43) Pub. Date: Oct. 5, 2017

(54) NEEDLE SURFACE FOR REDUCED COAGULATION AND METHOD FOR SAME

(71) Applicant: Cook Medical Technologies LLC, Bloomington, IN (US)

(72) Inventor: Pádraig M. Gunning, Co. Clare (IE)

(73) Assignee: Cook Medical Technologies LLC, Bloomington, IN (US)

(21) Appl. No.: 15/467,726

(22) Filed: Mar. 23, 2017

Related U.S. Application Data
(60) Provisional application No. 62/316,755, filed on Apr. 1, 2016.

(51) Int. Cl. A61L 33/00; A61L 33/04; A61B 10/02; A61B 8/08; A61B 10/04

(52) U.S. Cl. CPC A61L 33/0082; A61B 8/0841; A61B 10/04; A61B 10/0275; A61L 33/04; A61B 2010/045; A61L 2400/10; A61L 2420/02; A61L 2300/606; A61L 2300/452; A61L 2300/424; B05D 7/14

(57) ABSTRACT

A medical needle may include a lumen coating configured to reduce surface energy of the lumen-facing/lumen defining surface, in a manner effective to slow and/or reduce coagulation of biomaterial (particularly blood) that contacts the needle lumen surface. Such a coating may include a hydrophobic coating such as a silane and/or siloxane material on at least the needle lumen surface. The coating reduces a surface energy of coated needle regions below the surface energy of uncoated regions, and particularly reduces the polar component of the surface energy in the coated needle regions. The needle may be a metallic biopsy needle with the coating comprised by at least a distalmost length of sample-collection lumen.

### CROSS-REFERENCE TO RELATED APPLICATIONS

This application is a non-provisional application which claims priority to U.S. provisional application Ser. No. 62/316,755, filed Apr. 1, 2016, which is incorporated by reference herein in its entirety.

### TECHNICAL FIELD

Embodiments disclosed herein generally relate to medical devices including biopsy needles. More particularly, embodiments herein relate to needles including a lumen coating of a surface energy-reducing material.

### BACKGROUND

Biopsy and other needles have a variety of uses in diagnosing and treating human and non-human patients. For example, ultrasound-guided fine needle aspiration and core biopsy needles (including “fine needle biopsy” FNB needles) may be used for sampling from and/or delivering material to targeted submucosal extramural lesions, mediastinal masses, lymph nodes, and intraperitoneal masses within or adjacent to the tracheobronchial tree or the gastrointestinal tract via an accessory channel of an ultrasound endoscope. In fine needle aspiration, a small amount of fluid and cells are collected from a targeted area, while in fine needle biopsy, a core sample of tissue, cells, and fluid is collected from a targeted area. The former may be examined using cytopathology, and the latter may be examined using both cytopathology and histopathology for diagnosis of cell conditions and tissue conditions, respectively.

In sample collection techniques using these types of needles, the needle typically is directed through an accessory channel of an ultrasound endoscope to a target site (e.g., via a gastrointestinal endoscope through the stomach wall into a pancreatic lesion, or via a bronchial endoscope to a lesion of interest). The lesion is penetrated while vacuum/suction is applied through the needle lumen, and during which the needle may be manipulated in a variety of ways known to enhance sample acquisition and sample integrity. Then the needle is removed from the endoscope and the sample is ejected from the needle, typically using compressed air, cytology fluid or other fluid, or a long stylet.

A typical needle may be made of 304 stainless steel (a/k/a A2 stainless steel, outside of the United States), an austenite steel that has low electrical and thermal conductivity with desirable malleability and ductility and a composition that is highly corrosion-resistant generally including about 18% (that is about 17%-about 20%) chromium and about 8% (that is about 7% to about 11% nickel). The endoscopic needle configuration typically is greater than 0.5 m in length and often about 1 to about 1.7 m in length, with an inner diameter between about 0.37 mm and about 0.95 mm. The small diameter of the needle lumen combined with long needle length and relatively short coagulation time for typical samples (often less than one minute, and potentially only about 25-30 seconds) can make it difficult to remove samples from needles without sacrificing sample quality. This can result in extended procedure times due to a need for repeat sampling, insufficient sample sizes, user frustration, and less-than desired outcomes for procedures and diagnostic testing results.

Thus, it may be desirable to develop a needle that provides for decreased likelihood of rapid coagulation (e.g., of blood plasma and/or other bodily material) within a lumen of the needle and prevents occlusion of the needle lumen.

### BRIEF SUMMARY

In one aspect, embodiments disclosed herein may include needles that include a hydrophobic needle surface coating—particularly a needle lumen coating, as well as methods for preparing such needles.

In certain embodiments, a medical needle may include a tubular metallic needle body circumferentially defining a needle lumen including a lumen surface, where at least the lumen includes a hydrophobic coating along at least a portion of the lumen surface. The hydrophobic coating may include a silane such as (by way of nonlimiting example) dimethyldichlorosilane, a siloxane, or another hydrophobic coating that will reduce the needle's surface energy, and that particularly will reduce the polar component of the needle's surface energy, both with reference by way of comparison to an uncoated needle surface.

In further embodiments, needles of the present disclosure may include a side notch, a sample-receiving region (e.g., of the lumen), and/or other features wherein the needle is configured as a biopsy needle.

### BRIEF DESCRIPTION OF THE DRAWINGS

FIG. 1A shows a diagram of the human blood coagulation cascade;

FIG. 1B shows a diagram of siliconization of a glass surface;

FIG. 1C shows a diagram of siliconization of an oxidized metal surface;

FIGS. 2A-2B show two views of another tissue-sampling needle device embodiment;

FIG. 3 shows another tissue-sampling needle device embodiment; and

FIGS. 4A-4B show, respectively, magnified views of an uncoated needle surface and a coated needle surface.

### DETAILED DESCRIPTION

Various embodiments are described below with reference to the drawings in which like elements generally are referred to by like numerals. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as—for example—conventional fabrication and assembly. It should specifically be understood that each of the different coatings and methods described herein may be used with each of the different needles described herein.

The invention is defined by the claims, may be embodied in many different forms, and should not be construed as limited to the embodiments set forth herein; rather, these embodiments are provided so that this disclosure will be thorough and complete, and will fully convey enabling disclosure to those skilled in the art. As used in this specification and the claims, the singular forms “a,” “an,” and “the” include plural referents unless the context clearly dictates otherwise.

The terms “proximal” and “distal” are used herein to refer, respectively, to a handle/doctor-end of a device or related object and a tool/patient-end of a device or related object. The term “about” when used with reference to any volume, dimension, proportion, or other quantitative value is intended to communicate a definite and identifiable value within the standard parameters that would be understood by one of skill in the art (equivalent to a medical device engineer with experience in the field of medical needles), and should be interpreted to include at least any legal equivalents, minor but functionally-insignificant variants, and including at least mathematically significant figures.

Hydrophobicity and hydrophilicity are relative terms. A simple, quantitative method for defining the relative degree of interaction of a liquid with a solid surface is the contact angle of a liquid droplet on a solid substrate. When the contact angle of water is less than 30°, the surface is designated hydrophilic because the forces of interaction between water and the surface nearly equal the cohesive forces of bulk water, and water does not cleanly drain from the surface. If water spreads over a surface, and the contact angle at the spreading front edge of the water is less than 10° (high degree of wetting), the surface is often designated as superhydrophilic provided that the surface is not absorbing the water, dissolving in the water or reacting with the water. On a hydrophobic surface, water forms distinct droplets. As the hydrophobicity increases, the contact angle of the droplets with the surface increases. Surfaces with contact angles greater than 90° are designated as hydrophobic.

Hydrophobicity and hydrophilicity relate to the surface free energy (or, as interchangeably used herein, “surface energy”). The surface energy is a quantified measurement of disruption of bonds between molecules along a surface. Surface energy is expressed in units of force per unit length (e.g., N/m), or energy per unit area (e.g., J/m²). Surface energy may be described as having two energy components: dispersive energy which is attributable to van der Waals forces, and polar energy, which is attributable to all other intramolecular forces including dipole/dipole interactions, hydrogen bonding, it-cloud it-cloud interactions and so on. The term “surface tension” for surface energy is commonly used when discussing liquids, and the relative polar and dispersive components of surface energies of a liquid and a solid surface directly affect how they will interface when in contact. The more homogenous the relative polar and dispersive component of surface energies of a liquid and a solid surface, the better the wetting capability and thus the lower the contact angle between the two surfaces. It is well known that water has a high polar component to its surface energy, being about 72.8 mN/m (Dispersive 26.4 mN/m, Polar 46.4 mN/m), therefore a solid with a low polar energy component of its surface energy will be hydrophobic and there will be a large contact angle formed at the interface between the two surfaces.

Generally, needle embodiments disclosed herein include a lumen coating (though not limited to coating the lumen—as other surfaces may be coated also)—which coating may be disposed along at least a distalmost length of the needle lumen (e.g., distalmost at least 2 cm), where the coating provides for longer coagulation time as compared to an uncoated needle lumen with the same needle construction material. In particular, in each of the embodiments, the coating reduces the surface energy (both dispersive and polar components) of the coated needle surface to less than a surface energy of an uncoated surface of the same needle construction material.

It is widely recognized in the art that a parabolic relationship exists between the surface energy of a material and its procoagulant activity where optimal surface energies for biocompatibility (i.e., nondestructive interaction with biological sample materials) are generally understood to be between about 20 mN/m and about 32 mN/m. Surface energies above and below this general range are associated with increased bioadhesion, and—particularly for samples containing plasma or other bodily fluid subject to coagulation cascade activity—markedly reduced contact coagulation times, even though free surface energies between about 35 mN/m and about 50 mN/m may be suitable for needles, albeit less desirable than surface energies within the aforementioned range (particularly as surface energies above about 40 nM/m are considered to be in a zone of good bioadhesion). By way of illustrative example, certain biopsy needles tested (made of 304 stainless steel) had a surface energy of 43.48 mN/m (Disperse 36.16 mN/m, Polar 7.32 mN/m).

Of course bioadhesion is desirable in certain contexts, where one wishes a biological sample or other material to remain in place (e.g., on a glass slide for evaluation). In the context of glass containers for biological samples including blood plasma and whole blood, there is a well-developed literature regarding treating surfaces to avoid or minimize the interaction of the glass's surface with the biological material in a matter causing coagulation. Such methods have included providing active anticoagulant agents (e.g., heparin, or others) adhered, adsorbed, or otherwise bound to glass surfaces of containers or other glass materials that contact the biomaterial. The advent of fine needle aspiration and fine needle biopsy have led to recent innovations in sample collection technologies with regard to devices and methods, which has created a demand for further improvements. This includes a need for the ability to collect samples quickly and efficiently, but without being rushed/hurried by a need to get the sample out of the needle before it is rendered less useful or even useless due to rapid coagulation.

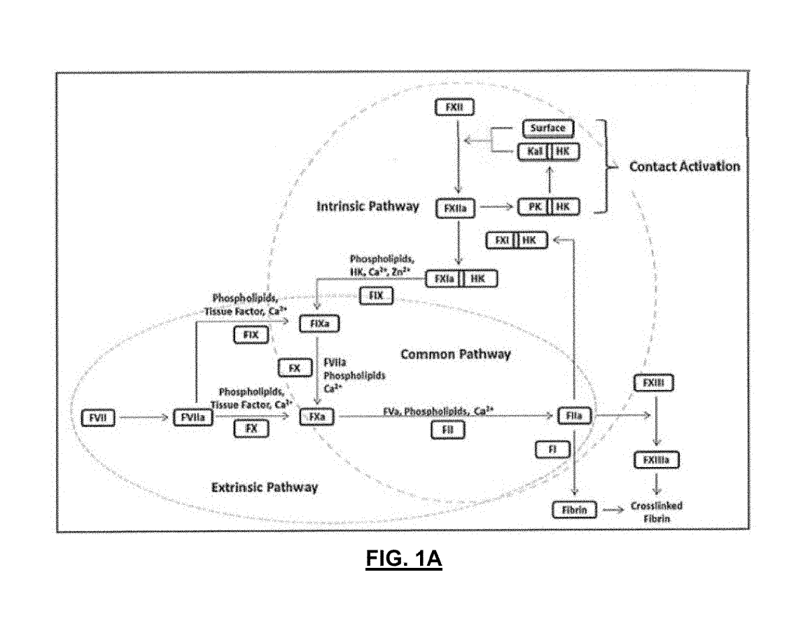

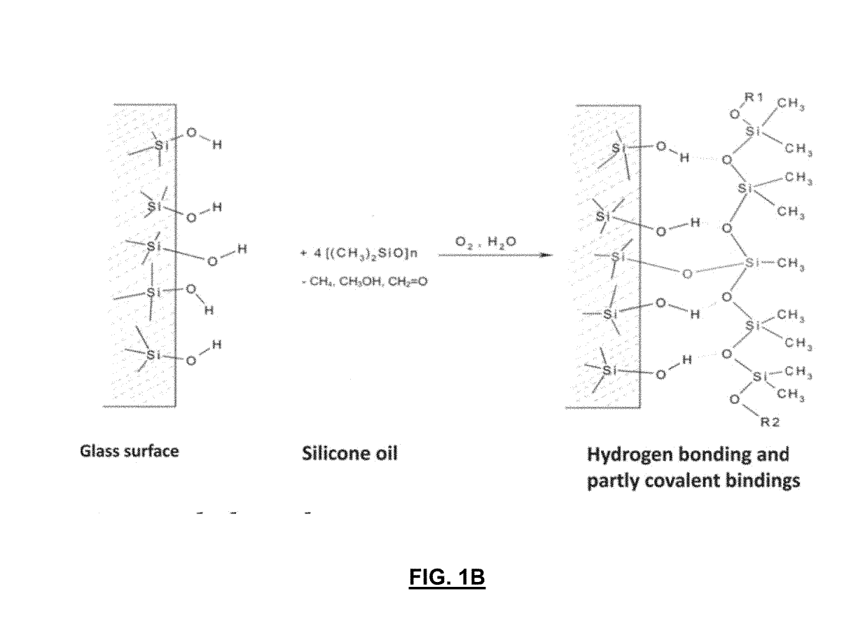

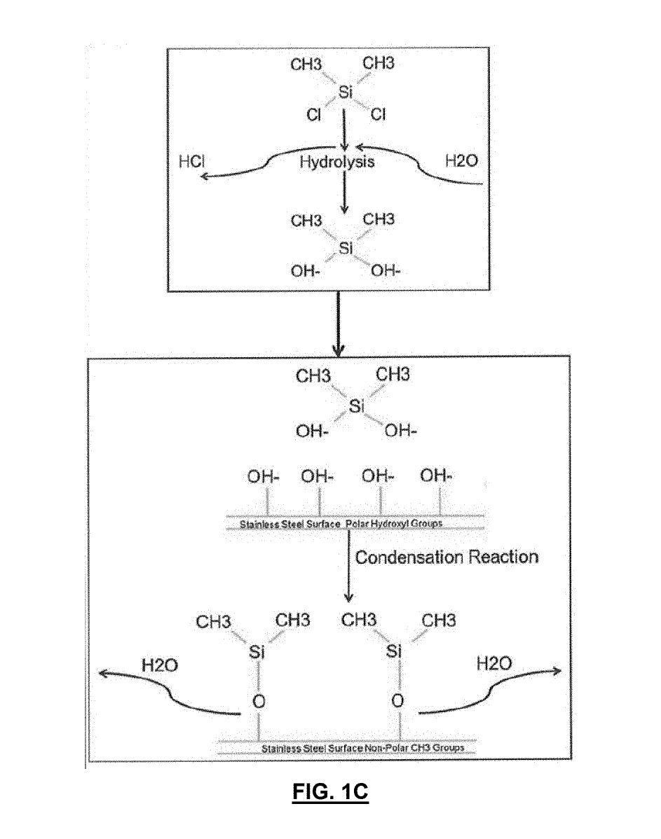

Coagulation in mammals, and particularly in humans, is fairly well characterized. In one simplified model, it can be portrayed that there are two main instigators of coagulation, as shown in FIG. 1A—contact activation and extrinsic factor activation. With contact activation, platelets or other components of whole blood (including, potentially, one or more plasma proteins may adhere or adsorb to the surface of a container or other substrate).

The inventors have discovered and developed structure and methods for reducing the surface energy within a needle lumen, particularly applicable to the inward-facing surface defining a lumen of biopsy needles but also useful for other applications. One example is coating at least a needle lumen portion, preferably a distalmost length likely to contact a biological sample being collected, with a material that reduces the free surface energy. Coating materials may include any material that reduces free surface energy and is suitable for coating a needle lumen (including most preferably with regard to being biocompatible and able effectively to coat a needle including a metal or polymeric needle). Particular coatings useful within the embodiments disclosed here may include silanes and/or siloxanes that most preferably are biocompatible and that provide for the surface energy property modifications described herein. The term “silane” is used herein to expressly include at least dimethyldichlorosilane, and generally to include other (most preferably biocompatible) hydrophobic organosilane compounds. The term “siloxane” expressly includes polysiloxanes.

By way of example for a stainless steel needle, it is noted that the surface of the needle lumen is an oxidized layer that includes a relatively high concentration of hydroxyl groups. The surface comprises polar oxide species that drive a high polar component in the stainless steel's surface energy and that increase the hydrophilicity of the surface. One exemplary silane, dimethyldichlorosilane (DMDCS), can be used as a needle surface coating to reduce surface energy. Through hydrolysis and a condensation reaction, the DMDCS replaces the stainless steel's polar surface hydroxyl groups with non-polar (hydrophobic) CH₃ groups, as shown in FIG. 1B. This is a somewhat different reaction than, and forms a somewhat different structure than when DMDCS is used to coat glass slides or containers, wherein silanol groups on the glass surface are deactivated. This is to say that, although glass is generally thought of as being inert, its surfaces are actually slightly acidic and highly adsorptive, so that they bind to biocommon molecules and groups (e.g., amine, carboxyl, hydroxyl, and thiol active groups) so that compounds in biological materials readily adsorb to untreated glass surfaces. Deactivation of the glass's adsorptive surface can be accomplished or at least made more effective by application of DMDCS, which complexes to provide a different surface chemistry as shown in FIG. 1C. The present method and structure are similar in that they also provide for reduced adsorption or other adhesion types of behavior, but the present method and structure also slows the procoagulative effect of blood plasma and/or other tissue sample components being in contact with needle surfaces, and particularly metallic needle surfaces.

The coatings described here include a variety of potential coatings that, when taught by the present disclosure, those of skill in the art will recognize as being useful to prevent, minimize, or at least slow down coagulation within needles used to administer, collect, and/or sample tissue or other biological materials. This includes FNA and FNB needles. Examples of such needles are disclosed in U.S. Pat. App. Publ. Nos. 2012/0253228, 2014/0257136, and 2015/0230780, each of which is incorporated herein by reference in its entirety. The surface properties of needles of the present disclosure (before coating) are different than the surface properties of glass described above and known in the art, and the differences in surface chemistry and structure between metallic needles (or even polymeric or ceramic needles) and glass surfaces known to be rendered less procoagulant were not predictive of the present discovery and development leading to the disclosure of hydrophobically coated needles herein that address a need in the medical field.

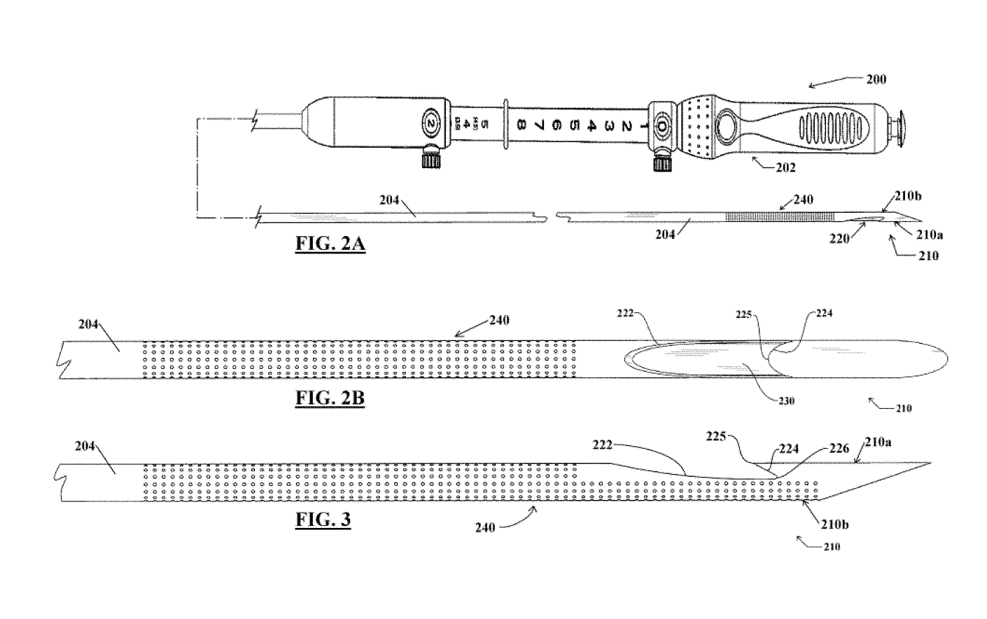

FIGS. 2A, 2B and 3 show embodiments of a tissue-sampling needle device 200, which may include a lumen coating of the present disclosure. As shown in the side plan view of FIG. 2A, the device includes a proximal handle or hub 202 from which an elongate tubular cannula 204 extends distally. The cannula 204 includes a tubular cannula wall that defines a cannula lumen, which preferably is uniformly cylindrical, but which may have an elliptical, obround, or other transverse cross-sectional profile, but which preferably is substantially uniform in its inner diameter (or other dimensions if non-circular in transverse cross-section) for at least a distalmost length that will contact and receive material being collected into the needle lumen, where—in the present embodiments—at least that distalmost length of inward-facing luminal surface most preferably will be coated with a material that provides a surface energy lower than uncoated luminal surface. A distal end 210 of the cannula 204 is beveled, including a long side 210a substantially parallel with the central longitudinal axis of the cannula 204 and extending to its distal-most tip end. A short side 210b of the beveled distal end 210 is opposite the long end 210a. A detail view of the needle device 200 is shown in a top plan view in FIG. 2B. The distal end 210 may be open to the lumen or may be closed. In embodiments with an open end 210, a sample may be ejected out the distal end after collection. In preferred embodiments disclosed herein, the sample will experience slower coagulation and less adhesion than a sample received into an uncoated lumen.

As shown in the side views of FIGS. 2A and 3, a notch 220 is disposed proximally adjacent to the beveled distal cannula end 210 and is generally centered in longitudinal alignment with the long beveled end side 210a and opposite the short beveled end side 210b. In preferred embodiments, the notch 220 is generally arcuate, defined on its proximal side by a parabolic edge 222 extending along generally longitudinal, but somewhat curved lateral notch sides 224. The distal edge 224 of the notch 220 preferably is formed as generally parabolic lip that joins the proximal edge 222 at a pair of lip end portions 226 that preferably provide a curved transition between the proximal and distal edges 222, 224. A central distal lip portion 225 of the distal edge 224 preferably forms a proximal-facing cutting edge. In preferred embodiments, the notch will occupy about one-half the circumference of the cannula 204 at the broadest point of the notch. In some embodiments, the notch 220 orientation may be reversed (that is oriented 180 different relative to the longitudinal axis, so that the parabolic lip faces distally rather than proximally). In preferred embodiments, the coating of the inward-facing luminal surface extends to at least a proximal end of the notch 220, and preferably extends more proximally therebeyond. In certain embodiments, the coating may extend at least 2× to 10× of that distance (from the distalmost needle terminus to the proximal end of the notch 220), and may extend up to the entire lumen length.

As shown in FIG. 2A, the cannula 204 includes exterior surface features 240 configured to enhance echogenicity, thereby providing an improved ability to navigate the device during an EUS procedure. The surface features 240 are shown here as dimples on an exterior surface of the cannula 204, but may alternatively be embodied as grooves or other regular or irregular features on an external or internal surface. Embedded echogenic features such as bubbles, voids, or pieces of echo-contrasting materials may also be used within the scope of the present invention. Those of skill in the art will appreciate that many currently-known and/or future-developed echogenicity-enhancing means may be used within the scope of the present invention. As used herein, the terms echogenic and echogenicity-enhancing are used to refer to structural features that increase the reflectivity of ultrasound waves used during ultrasound visualization of a device, with the increase being over the typical ultrasound reflectivity/visualizability of a device lacking the features described.

FIG. 3 is similar to FIG. 2A, but shows that the echogenic features 240 may extend distally across the space occupied by the notch 220. It is preferable that echogenicity-enhancing features be disposed at a specified predetermined distance from the distal-most tip end of the cannula 204. Although the echogenic features 240 are shown at a distance from the notch 220, a cannula according to the present embodiments may be constructed with those echogenic features disposed flush up to the margins of the notch. A stylet 230, which may include echogenicity-enhancing features may be disposed through the cannula lumen of the embodiments of FIGS. 2A-3. The coating described herein may ease the passage of the stylet into and out of the coated needle luminal region as well.

### EXAMPLE

A sample set of needles was prepared and tested in keeping with principles of the present disclosure. The test needles and control needles were identical, but for the coating of the lumen of the test needles. All needles were 304 stainless steel endoscopic biopsy needles.

The method of treating the test needles for coating included steps of ultrasonically cleaning the needles in ethanol for about 2 hours; removing and air-drying the needles; placing the needles into a coating solution (1.5 μL DMDCS with 500 μL ethanol, although other embodiments may use different concentrations, optimized in view of the present disclosure and informed by known properties for a particular needle material) on a mixing table for about 2.5 hours at about 120 RPM; removing the needles and rinsing them with ethanol; drying the needles in an oven at about 80° C. for about 6 hours; washing the needles with ethanol, and then finally air-drying them. Each of the time lengths and other particular aspects of the method steps above may be modified with regard to known properties of the materials being used, when informed by the present disclosure. The times, temperatures, and concentrations will be predetermined based upon the specific materials being used, and preferably optimized, none of which is expected to require undue experimentation in view of the present disclosure, the state of the art, and known/well-characterized needle materials. In an alternative method, only the inner lumen may be coated (e.g., by masking the exterior, by contacting only the needle lumen with the coating solution, or by other means known or developed in the art).

The uncoated/untreated needles have a surface elemental composition including 0.75% Si. Upon spectroscopic analysis, the surface elemental composition of the coated/treated needles included 3.72% Si, demonstrating effective siliconization.

An assay for physiological effectiveness of the coating in an in vitro simulated environment was developed and used to compare contact activation of the intrinsic coagulation pathway (see FIG. 1A). A physiologically-representative assay of human whole blood coagulation was implemented with a mixture comprising citrated platelet-poor plasma (human), partial thromboplastin derived from egg, and calcium chloride. The mixture was exposed to contact with the surface materials being investigated, and the time for coagulation was recorded. Coagulation time was noted by a distinct change in fluid-like rheology to gel-formation that determined the coagulation end point.

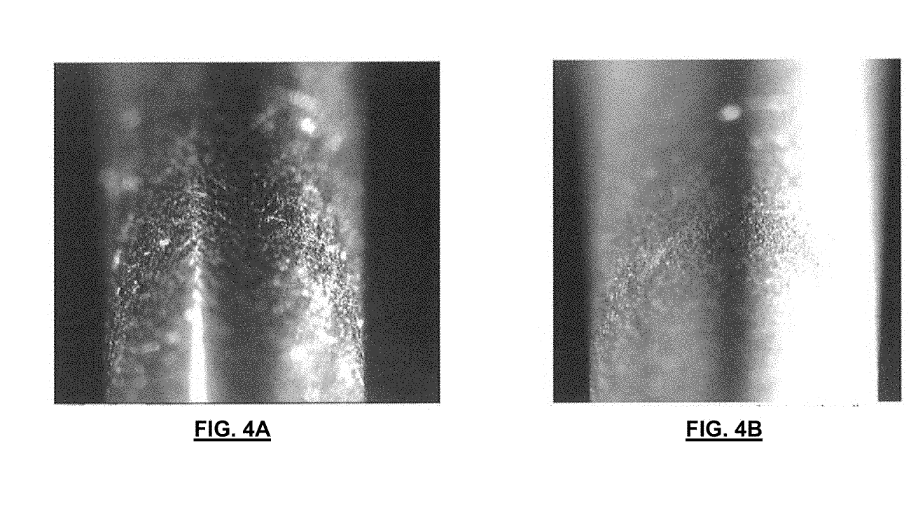

Uncoated needle surfaces had an average coagulation time of about 13 minutes, while the coated needle surfaces had coagulation times between about 34 minutes and about 12 hours. This clearly demonstrated a successful and noteworthy lengthening of coagulation time, which will improve outcomes for actual use of coated needles. FIGS. 4A and 4B show identically-magnified images of, respectively, and uncoated needle surface and a coated needle surface. The image shown in both of FIGS. 4A-4B at 124× magnification illustrates a metallic stainless steel needle surface. As evidenced by a visual comparison, the coated needle surface of FIG. 4A is smoother than that of the uncoated needle surface of FIG. 4B. The uncoated needles had a surface energy of about 43.48 mN/m (Disperse 36.16 mN/m, Polar 7.32 mN/m). However, the coated needles had a surface energy of about 29.83 mN/m (Disperse 29.62 mN/m, Polar 0.21 mN/m), which is well within the biocompatible range of about 20 mN/m to about 32 mN/m, and well below the bioadhesion range that is above about 40 mN/m. The low polar component of the coated needle also results in a hydrophobic surface with a water contact angle of about 113°.

As such, it should be appreciated that, according to the present disclosure, one may provide a tubular needle body circumferentially defining a needle lumen including a lumen surface. The lumen may be coated to include a hydrophobic coating along at least a distalmost portion of the lumen surface, where the hydrophobic coating provides a surface energy of the coated lumen surface that is between about 20 mN/m and about 32 mN/m, with a polar component that is at or lower than about 3 mN/m and which surface energy (both total surface energy and polar component portion) is lower than of an uncoated lumen surface. The coating may be a silane, a polysiloxane, or other appropriate hydrophobic coating (including any combination thereof), with one exemplary coating including DMDCS. The needle may be configured as a biopsy needle, but is not limited to such, as needles configured for introducing or transferring biomaterials (e.g., bone marrow, intact or disaggregated tissue and/or cellular material, or other biological materials) may also benefit from the reduced bioadhesion and reduced procoagulation properties of needles according to the present disclosure. A silane or other coating may provide a surface energy of the coated lumen surface that is at or below 30 mN/m and greater than about 20 mN/m with a polar component that is at or below about 3 mN/m. The coating preferably provides a surface energy of the coated lumen surface that is below about 32 mN/m with a polar component that is at or below about 3 mN/m. The tubular needle body may be metallic in preferred embodiments, but polymeric, ceramic, and/or other materials subject to coating with hydrophobic coating, and particularly coating with silicon-based material (especially silane and/or siloxane) may be used in a needle body—alone, combined with each other, and/or combined with a metal body.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation unless specifically defined by context, usage, or other explicit designation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A medical needle, comprising:
a tubular metallic needle body circumferentially defining a needle lumen including a lumen surface;
said lumen comprising a hydrophobic coating along at least a portion of the lumen surface.

2. The needle of claim 1, where the hydrophobic coating comprises silane.

3. The needle of claim 2, where the silane coating comprises dimethyldichlorosilane.

4. The needle of claim 2, where the needle is configured as a biopsy needle, and the silane coating covers the lumen surface of at least a distalmost length of the needle.

5. The needle of claim 2, where the silane coating provides a surface energy of the coated lumen surface that is less than a surface energy of an uncoated lumen surface.

6. The needle of claim 1, where the hydrophobic coating provides a surface energy of the coated lumen surface that is between about 20 mN/m and about 32 mN/m.

7. The needle of claim 1, where the hydrophobic coating provides a surface energy of the coated lumen surface that is below about 32 mN/m.

8. The needle of claim 1, where the hydrophobic coating provides a surface energy of the coated lumen surface that is at or below 30 mN/m and greater than about 20 mN/m.

9. The needle of claim 1, where the hydrophobic coating provides a surface energy of the coated lumen surface that has a polar energy component that is at or below 3 mN/m.

10. The needle of claim 1, where the needle is configured as an endoscopic biopsy needle, at least a distalmost 2 cm of the needle lumen comprises the hydrophobic coating, and the needle is configured with flexibility and dimensions for operation through an accessory channel of a surgical endoscope.

11. The medical needle of claim 1, where the needle includes a side notch, and the coating covers at least a distalmost length of the needle between a distal terminal needle end and a proximal end of the notch.

12. A medical needle, comprising:
a tubular needle body circumferentially defining a needle lumen including a lumen surface;
said lumen comprising a hydrophobic coating along at least a distalmost portion of the lumen surface,
where the hydrophobic coating provides a surface energy of the coated lumen surface
that is between about 20 mN/m and about 32 mN/m, and
that has a polar energy component that is at or below 3 mN/m, and
that is lower than an uncoated lumen surface.

13. The needle of claim 12, where the hydrophobic coating comprises a silane, a siloxane, or any combination thereof.

14. The medical needle of claim 12, where the needle includes a side notch, and the hydrophobic coating covers at least a distalmost length of the needle between a distal terminal needle end and a proximal end of the notch.

15. The needle of claim 12, where the tubular body is metallic.

16. A method of making a needle according to claim 15, said method comprising steps of:
cleaning a tubular metallic needle body in ethanol;
placing at least a portion of the needle body into a coating solution comprising dimethyldichlorosilane for a predetermined time; and
removing the needle body from the coating solution before drying them in an oven at a predetermined time and temperature.

17. The method of claim 16, further comprising a step of washing the needle body with ethanol.

18. The method of claim 17, further comprising a step of air-drying after the step of washing.

19. The method of claim 16, where the metallic needle body is configured as a notched needle.

20. The method of claim 16, where the step of placing the needle into coating solution comprises placing less than the entire needle into the coating solution.
